(19) United States
(12) Patent Application Publication
(10) Pub. No.: US 2018/0297037 A1
(43) Pub. Date: Oct. 18, 2018

(54) METHOD AND APPARATUS FOR CONVERTING INFECTIOUS WASTE MATERIAL INTO MATERIAL USABLE AS FUEL FOR A CEMENT KILN

(71) Applicant: John R. Self, Cullman, AL (US)

(72) Inventor: John R. Self, Cullman, AL (US)

(21) Appl. No.: 15/487,250

(22) Filed: Apr. 13, 2017

(51) Int. Cl. B02C 19/00; B09B 3/00; A61L 11/00; C10L 5/46; B02C 19/22

(52) U.S. Cl. CPC B02C 19/0075; B09B 3/0075; B09B 3/0083; C10L 2290/24; C10L 5/46; B02C 19/22; C10L 2290/32; A61L 11/00

(57) ABSTRACT

An infectious waste treatment system and method for decontaminating infectious waste employ a thermal friction extruder (20) in which first and second interleaved counter-rotatable augers (40, 42) driven by a variable speed motor include reverse pitch flight sections (62, 72) that urge waste material in a direction opposite to that of the flow stream and into engagement with the back sides of friction plates (50, 52). This increases the amount of heat generated by the extruder. The reverse pitch flight sections can be selectively replaced with forward pitch flight sections to control the amount of heat imparted to the waste material by the friction plates. The size of gaps between the friction plates and the augers is selected along with the motor speed to impart enough heat and friction to the waste material, such that the resulting processed material has an increased BTU value, a consistency and aggregate size such that it can readily be injected as fuel into a cement kiln's fuel injection system, thereby completely disposing of the material.

### TECHNICAL FIELD

The present invention relates generally to the field of processing contaminated infectious waste material in which internal friction is used to convert the waste to a material that is suitable for use as fuel in a cement kiln. The method and apparatus are particularly suited for the thermomechanical decontamination and volume reduction of infectious medical waste, wherein the resulting material can be reclassified as a material that can be used as fuel for use in a cement kiln.

### BACKGROUND ART

In the decontamination of infectious waste, namely medical waste, it is important to insure that the ultimate waste product, which, up until now, was to be discarded, is free of pathogenic microorganisms. It is also highly desirable, and in some instances required by law, to render infectious waste in a condition such that individual components, such as disposable syringes, bandages, body fluid receptacles, and even body parts removed in surgery or in autopsies, are unrecognizable.

Infectious waste such as medical waste is generated by hospitals, medical laboratories, and the like and is required to be decontaminated prior to being disposed. Examples of medical waste include hypodermic syringes, glassware, slides, gauze, needles, infectious tissues, blood-soaked materials, red bag waste, or other such potentially infected or contaminated medical waste materials typically generated during the normal operation of a hospital, medical laboratory, or the like. Public; concern over the proper treatment and disposal of medical waste products has increased over the past several years. This increase is due in part to an increased public awareness of the diseases that can be transmitted by biologically contaminated waste products. It is therefore desirable to produce a disposal system which adequately disinfects infectious waste products while rendering the waste unrecognizable to the degree that it can be disposed of in an approved disposal facility and/or used as a fuel source without posing further infection threats due to contact with the post-treated residual waste.

The prior art has attempted to address the problem of disposing of medical waste by methods such as specialized land filling, incineration, steam autoclaving, chemical treatment, and/or radiation treatment. Environmental regulations have severely limited the use of incineration for infectious waste disposal due to the potential production of gaseous emissions that may contain high levels of toxic heavy metals, e.g. cadmium, chromium, lead, mercury, dioxins and furans generated by the plastics and metallic content derived from syringes, needles, and sharps included in the waste. In addition, incinerators are not fully satisfactory because they require regular servicing and cleaning.

Steam decontamination is another known method for treating medical waste. Steam decontamination is primarily performed in steam autoclaves. Steam autoclaving is a thermal process in which the wastes are disinfected by exposure to high-temperature steam and pressure. The high temperature and good penetrability of steam effectively destroys the infectious agents. Since the waste is rendered disinfected, it can be directly landfilled. However, for steam autoclaving to be an effective treatment method, the steam must fully penetrate the waste to ensure that all infectious microorganisms are destroyed. Also, since autoclaved waste is neither mechanically destroyed nor significantly reduced in volume, it is still recognizable as medical waste and treated hypodermic needles still pose a stick threat.

Still another method is the chemical decontamination of infectious waste. Hospitals and other health care facilities have used chemical agents routinely for decades in the decontamination of infectious waste. As in steam autoclaving, chemical decontamination will not be effective unless there is adequate contact between the infectious waste and the chemical. In addition, the chemical should be maintained at a sufficient concentration and there should be sufficient exposure time between the waste and chemical to achieve proper levels of decontamination. There are several other disadvantages of using chemicals in the decontamination of infectious waste including potential occupational exposures of workers to chemical concentrations in the air and through skin contact; the possibility of toxic byproducts in the wastewater; chemical hazards involved with the use and storage of the chemicals; chemical residue in the treated waste; and offensive odors.

Still another method of disinfecting infectious wastes is to use radiation treatment. The radiation may be microwave frequencies, shortwave radiofrequencies, and the like. The radiation treatment suffers from several disadvantages. First, radiation treatment by itself will not render the waste unrecognizable. Second, radiation involving the use of microwaves is not suitable for treating chemotherapy wastes or human organs or body parts. Third, the infectious waste must have a significant moisture content to insure effective treatment with microwaves.

A more promising approach for decontaminating medical waste is to use a thermal friction extruder apparatus using friction generated by counter-rotating interleaved worm gear augers or screws to grind and heat the waste. The basic concept of using counter-rotating screws to impart heat-generating friction to a material is disclosed, for example, in U.S. Pat. No. 4,599,002, which issued on Jul. 8, 1986 (hereinafter, “the '002 patent”). This patent specifically discloses a screw extruder for compressing a material in which two interleaved counter rotating screw or auger members extend through a plurality of casing members, each separated by a corresponding one of a plurality of throttle plate members. The throttle plate members each has an orifice there through of a preselected dimension that is selected to effectively block the free-section of the screw members whereby, material to be compressed is pressed against the throttle plate member with a large force thereby causing heat that creates pulverization and drying of the material by friction on each of the throttle plate members. However, the '002 patent apparatus is not designed to decontaminate infectious waste and would not be able to generate enough heat to do so without some kind of modifications.

International Patent Application No. PCT/US2011/042905, which was published on Jan. 5, 2012 as International Publication Number WO 2012/003507 A1 (hereinafter, “the 507 application”) discloses a system which employs a thermal friction extruder apparatus similar in construction to the extruder disclosed in the '002 patent, to decontaminate infections waste. To increase the amount of heat generated in the extruder, an adjustable outlet size extruder die is located at the terminal end of the apparatus for converting the ground medical waste into an extrudate and controlling the backpressure of the extrudate as it exits the extruder. The extruder die has an adjustable outlet valve piston disposed therein for controlling the size of and therefore the temperature of the extrudate. The extruder die can thus be employed arguably to impart enough heat to the waste that it will be decontaminated.

However, in practice, to insure that the amount of heat generated by the extruder is sufficient to raise the temperature of the waste high enough to disinfect the same, it has been found that the size of the extruder die has to be adjusted so small, that it becomes too difficult to move the material through the extruder die. In addition, the tremendous backpressure imparted to the material stream by the extruder die subjects the apparatus to excessive wear and tear.

International Application Number PCT/US2015/014903, filed on Feb. 7, 2015 to John R Self et al and published on Aug. 13, 2015 as International Publication Number WO 2015/120323 A1 (hereinafter, “the '323 application”) is directed to a thermal friction extruder type infectious waste treatment system and method for the decontamination of infectious waste that overcomes the forgoing drawbacks of the apparatus disclosed in the '507 application.

More particularly, the thermal friction extruder used in the '323 application is similar in construction to those disclosed in the '002 patent and '507 application with a particularly notable exception that reverse pitch auger sections are selectively employed to urge waste in a direction opposite to that of the flow stream and into contact with the back sides of one or more friction plates. The reverse pitch auger sections cause an increased amount of heat to be generated in the infectious waste without the need for a backpressure inducing extruder die at the discharge end of the extruder. As a result, the extruder can operate without an extruder die and the resulting excess stress being applied to the apparatus and without impeding the free flow of waste material through the extruder.

Preferably, the extruder includes multiple sections referred to as compression chambers that are separated from one another by a corresponding plurality of friction plates. As in the '002 patent and the '507 application, the friction plates are designed to form a small gap between the auger flight outer diameters and the friction plate inner diameters. This gap enables waste material to pass there through only once the waste has been reduced in size to the size of the gap. Each of the auger members includes small aggressively pitched auger sections that are positioned adjacent each friction plate on both sides thereof. The aggressive auger sections in the first chamber are positioned adjacent to the front side of the first friction plate and act to push the waste material into engagement with the friction plate front side. After the material passes through the gap between the first friction plate and the auger flights, the materiel engages a reverse pitch auger section that urges the waste material back in a direction opposite to that of the flow stream and into contact with the back side of the first friction plate. This causes further frictional heating of the waste material.

The foregoing process is repeated in a second and even additional compression chambers until the waste reaches a temperature where it is fully decontaminated. The use of a series of reversed pitch auger sections, which can be effectively added or subtracted by simply swapping the interleaved reverse pitch sections in the two counter-rotating augers, to produce more of less frictional heat thus enables control of the internal temperature of the friction extruder.

It has now been established that the use of the device disclosed in the '323 application does in fact disinfect infectious medical waste to the extent that the waste material can be classified as decontaminated medical waste and disposed of in one of two approved ways, either in; 1) a landfill, or 2) as fuel in a waste-to-energy plant designed to burn municipal solid waste. There are significant drawbacks to these approved disposal methods. Landfilling does not make the waste go away. It will remain interned for centuries giving off greenhouse gases and contributing to climate change. Burning in a waste-to-energy plant is a financial burden to the medical waste industry. These plants charge to burn the treated waste, in some cases $75.00 per ton. Additionally the resulting ash is buried in an associated landfill. Thus, the treated medical waste never goes away completely.

Now that a successful process has been devised to decontaminated infectious medical waste, a need therefore remains for a way in which the material can be completely disposed of and preferably with a minimum of expense.

### SUMMARY OF THE INVENTION

Applicant has now discovered that the system disclosed in the '323 application can be made to generate treated medical waste that can be used as fuel specifically in a cement kiln. Cement kiln fuel arguably is the perfect disposition for treated medical waste. This is because cement kilns burn the fuel and use 100% of the remaining ash as aggregate in the cement product. Nothing is ever landfilled or discarded.

However, there are obstacles in implementing this preferred disposition technique. Most States, including Florida, have antiquated medical waste laws and require, by law, that all treated medical waste either be buried in a landfill or burned in a municipal waste-to-energy plant. A variance is required by the State DEP to get around this law.

All these laws were written with autoclaves in view. Previously, autoclaves were the prevailing or only available technology for the treatment of medical waste. However, autoclaved medical waste presents a post treatment hazard due to the fact that the waste has not undergone any physical change and looks just like it did pre-treatment. As a result, in Florida, for example, no waste-to-energy plant will accept autoclaved medical waste and only a couple of landfills will accept it.

The States classify treated medical waste as municipal solid waste, (it is no longer infectious) but with the caveat that it is treated medical waste and must be labelled as such. Many cement kilns are not permitted to burn municipal solid waste because their air permit will not allow its use. The solution is to have the treated medical waste classified as something other than municipal solid waste.

The EPA came up with a solution by forming a new fuel classification for material that was once classified as waste. This new classification is the result of the implementation of the Non Hazardous Secondary Material (NHSM) regulation. For a material to qualify as an NHSM it must meet three criteria:

1) It must be processed in such a way that the material's BTU value is enhanced, i.e. homogenized and DRIED! The simple act of shredding does not satisfy this rule!

2) There must be a customer willing to purchase it as fuel.

3) The contaminants in the new fuel must not be higher than the proposed natural fuel (i.e. coal) that it is replacing.

The thermal friction extrusion technology disclosed in the '323 application can accomplish all of the above 3 necessary criteria. ONLY the thermal friction extrusion technology disinfects medical waste while grinding it into a fine fluff and eliminating the majority of the moisture content. Lab testing shows that the thermal friction extrusion treated medical waste has s BTU value of 13,000/lb. In contrast, autoclaves and other treatment technologies ADD moisture to the waste for treatment.

One cement manufacturer, after close examination of the material generated using the subject system and process, offered to purchase the same for a substantial sum of money. This is because the treated material is dry and so finely ground that it can be blown into the burners of their cement kilns. This could not be done with shredded material, for example. Thus, the second criterion above is satisfied.

Lab testing also shows that medical waste treated via thermal friction extrusion does not contain more contaminants that coal, thus satisfying the 3^{rd} EPA criterion for reclassification as NHSM.

Once classified as NHSM, the treated medical waste generated by the subject system and process would be available for any cement kiln use and not affect their air permit because it is no longer classified as municipal solid waste.

Thus, in this application, the thermal friction extrusion treatment process is thus the key to eliminating medical waste from the face of the planet forever!

The only thing needed to adapt the system for use in converting infectious medical waste to cement kiln fuel is that it needs to be modified somewhat in order to meet the fuel requirements of cement kiln operators.

Mechanical modifications needed to produce NHSM acceptable and cement kiln acceptable for sale as a commodity to a cement kiln include the following:

1) The first friction plate immediately after the feed chamber must be open 4.5 mm+−0.5 mm. This ensures that sufficient material enters the compression chamber to generate disinfection heat but not too much to generate too much heat that would cause the material to melt and form hard plastic briquettes.

2) The second and last friction plate must be open at least 1.5 mm to allow material to exit but not more than 2.5 mm which would produce material size too large for use as NHSM in cement kilns.

3) Auger flights immediately in front of each friction plate must be configured with at least 4 and no more than 5 flights set at 1.5 rotations in pitch. This insures that the material is sufficiently ground against both friction plates to produce the small aggregate required for use in a cement kiln.

4) Gear rotation speed must be maintained at 90 RPM+−5%. Greater speeds would melt the plastic and less would not create enough heat for disinfection.

The system disclosed in the '323 application also satisfies two other requirements as is. The first is that no exit die can be used. A die would eventually generate a briquette and ruin the material for use as NHSM cement kiln fuel. The second is that the system must have the ability to reverse the flights downstream of the friction plates to carefully regulate the internal temperature of the waste, which is an existing characteristic of the system in the '323 application. The waste must never be allowed to reach a melting point.

### BRIEF DESCRIPTION OF THE DRAWINGS

The foregoing and other features and advantages of the present invention will become apparent from the following detailed description of a preferred embodiment thereof, taken in conjunction with the accompanying drawings, which are briefly described as follows.

FIG. 1 is a schematic illustration of an infectious waste treatment system that is configured in accordance with a preferred embodiment of the present invention.

FIG. 2 is a cross sectional schematic of a preferred embodiment of a thermal friction extruder that is employed in the preferred embodiment of the invention.

FIG. 3 is a cross section of the extruder's auger roots as they pass through a friction plate.

### DETAILED DESCRIPTION OF THE PREFERRED EMBODIMENT

The previously discussed prior art references, U.S. Pat. No. 4,599,002, issued Jul. 8, 1986; Published International Application No. WO 2012/003507, published on Jan. 5, 2012; and, Published International Application No. WO 2015/120323, published on Aug. 13, 2015, are each hereby incorporated by reference in their entireties.

As noted above, the present invention relates to a system and method for the thermomechanical treatment of infectious waste wherein, using friction as the sole source of decontamination, the infectious waste is not only rendered decontaminated and unrecognizable, but in addition, acceptable for use as a fuel in a cement kiln. “Infectious waste” shall generally be defined as any material that is capable of producing disease. The definition of infectious waste shall include but shall not be limited to medical waste wherein “medical waste” is defined as any solid waste generated in the diagnosis, treatment, or immunization of human beings or animals, in research pertaining thereto, or in the production or testing of biologicals, excluding hazardous waste identified or listed under 40 CFR Part 261 or any household waste as defined in 40 CFR Sub-section 261.4 (b) (1). “Decontamination” means either the substantial sterilization or disinfection of infectious waste. “Sterilization” means the removal or destruction of all microorganisms. “Disinfection” is a somewhat less lethal process than sterilization which destroys or inactivates viruses, fungi, and bacteria (but not necessarily their endospores) on inanimate surfaces. “Unrecognizable” means that the original appearance of the feed material has been altered such that neither the feed material nor its source can be identified. “Thermomechanical” means the combination of friction and mechanical deformation.

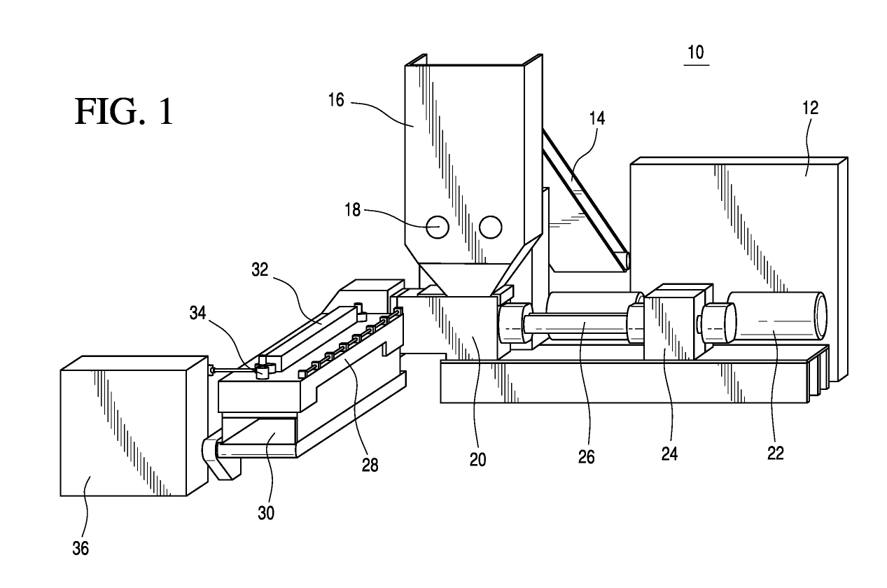

A decontamination system 10 which is configured in accordance with a preferred embodiment of the invention is shown in FIG. 1. The decontamination system 10 is structurally the same as the system disclosed in the '323 application except for some notable exceptions. As a result, much of the system 10 need not be discussed in detail here and the reader is invited to refer to the '323 application in which the entire system is discussed in greater detail Only the parts of the system 10 that are particularly relevant to the conversion of the medical waste to cement kiln fuel will be discussed in detail here.

As in the system disclosed in the '323 application, the system 10 is designed to treat infectious waste that is introduced into the system whereby the waste will be decontaminated and rendered unrecognizable. In addition, and as required for use in a cement kiln, the consistency of the treated material is such that it can be easily introduced through a fuel injection system of a conventional cement kiln and thereby used as fuel therefore. This precludes the formation of large briquettes or large shredded pieces that could not be injected into a cement kiln through the kiln's standard fuel injection system. (It should be noted that this should not be confused with known cement kilns that have been substantially modified to act as incinerators which burn most any type of waste material.)

The resulting treated waste material has a BTU value that is higher than that of the original untreated waste and contains less contaminates than coal, which is a comparable fuel. Thus, the EPA will allow the waste generated by the system 10 to be reclassified as a NHSM and cement kiln operators will be free to buy and use the material.

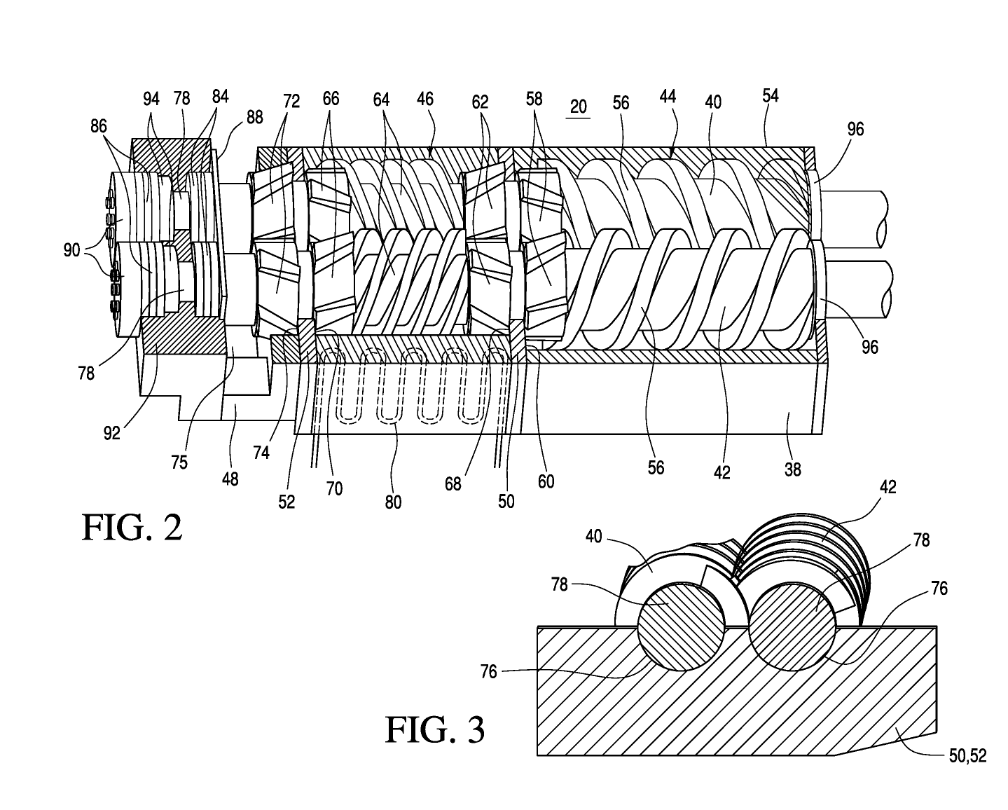

In the overall operation of the treatment system 10, the waste is fed into a thermal friction extruder 20 which is the key component of the treatment system 10 and serves to thermomechanically decontaminate the waste and render the same unrecognizable. The extruder 20 is discussed in greater detail in conjunction with FIGS. 2 and 3. An electric motor 22, transmission 24 and drive shafts 26 provide and transmit power to rotate screw augers in the extruder 20.

With reference to FIG. 2, a sectional view of the thermal friction extruder 20 is illustrated which shows the various internal elements of the same that are disposed in an extruder housing 38, which is shown partially cut-away in FIG. 2. The extruder 20 includes first and second interleaved counter-rotatable augers 40 and 42, each of which extends through first and second compression chambers 44 and 46; and then terminate in a thrust bearing assembly housing 48. It should be noted, however, that the extruder 20 may include any number of compression chambers. The number of compression chambers can be adapted to the material to be processed.

The first and second compression chambers 44 and 46 are separated from one another by a first friction plate 50, while the second compression chamber 46 and the thrust bearing assembly housing 48 are separated by a second friction plate 52. Waste to be decontaminated that is received from the feed hopper 16 enters an inlet end 54 of the extruder 20 in the first compression chamber 44. Disposed in the first compression chamber 44 are first and second pairs of flight sections 56 and 58 of each the augers 40 and 42, with the second sections 58 preferably being aggressive 4″ section of flights which grind and force the waste into a front side 60 of the first friction plate 50. The second sections 58 must be also configured with at least 4 and no more than 5 flights set at 1.5 rotations in pitch. This insures that the material is sufficiently ground against the friction plate 50 to produce the small aggregate required for use in a cement kiln.

Eventually, the waste is ground down enough that it fits through a gap formed between the friction plate and the flights of the augers 40 and 42 as illustrated in FIG. 3, which is discussed in greater detail below.

The second compression chamber 46 contains first, second and third flight sections 62, 64 and 66 of each of the augers 40 and 42. The first flight sections 62 are positioned adjacent to the back side 68 of the first friction plate 50. As illustrated, the flights of first section 62 are reverse direction pitch flights, which force the waste back against the back side 68 of the first friction plate 50, thus increasing the thermomechanical heat being developed. The flights of the first flight sections 62 are designed with flight pitches shallow enough to create backpressure toward the back side 68 of the first friction plate 50, but are not aggressive enough to stop downstream movement of the waste material. The preferred flight pitch of auger sections 62 is 15° to 35°.

The second and third flight sections 64 and 66 in the second compression chamber 46 include forward or downstream direction flights that move the waste toward the second friction plate 52. As with the second flight sections 58 in the first compression chamber 44, each of the third flight sections 66 includes an aggressive 4″ section of flights which grinds and forces the waste into and eventually under a front side 70 of the second friction plate 52. This section of flights must be also configured with at least 4 and no more than 5 flights set at 1.5 rotations in pitch. This insures that the material is sufficiently ground against the second friction plate 52 to produce the small aggregate required for use in a cement kiln.

The thrust bearing assembly housing 48 contains a pair of final flight sections 72 which are positioned adjacent to the back side 74 of the second friction plate 52. As with the flight sections 62, the flights of final flight sections 72 are also reverse pitch direction flights, which again force the waste back against the back side 74 of the second friction plate 52, thus further increasing the thermomechanical heat being developed. as with fight sections 62, the flights of the final flight sections 72 are designed with flight pitches shallow enough to create backpressure toward the back side 74 of the second friction plate 52, but are not aggressive enough to stop downstream movement of the waste material. The preferred flight pitch of auger sections 72 is 15° to 35°.

Both of the pairs of auger flight sections 62 and 72 that are shown with reverse direction flights are preferably designed to be reversible so that the flights also can be positioned in the downstream direction if desired. Since the interleaved counter-rotating augers 40 and 42 inherently have auger sections which are of opposite pitch to one another as shown, the direction of the flights in the reverse pitch sections 62 can easily be reversed by simply swapping the section 62 on the first auger 40 with the section 62 on the second auger 42. This versatility enables the temperature of the waste material in the extruder 20 to be more precisely controlled, especially in the case where two or more compression chambers and corresponding friction plates are employed. In this regard, one or more temperature sensors (not shown) are preferably disposed in the extruder housing 38.

After the now decontaminated waste passes through the final reverse direction flight sections 72, the waste is discharged through an outlet 75 and deposited onto the conveyor 30 of the extended residence chamber 28 shown in FIG. 1.

A preferred embodiment of the auger root and friction plate arrangement is shown in FIG. 3. A gap (not visible in the drawing) is formed in the interface 76 between the outside diameter of the auger root 78 and the inside diameter of each of the friction plates 50 and 52. This gap 76 is selected to be big enough to allow grinded and crushed waste to pass through but small enough so that unground waste will not pass through. In the specific application of the system 10 to make cement kiln fuel, the gap size of the first friction plate 50 must be 4.5 mm+−0.5 mm. This ensures that sufficient material enters the compression chamber to generate disinfection heat but not too much to generate too much heat that would cause the material to melt and form hard plastic briquettes that could not be used as a fuel in a cement kiln. As for the second or last friction plate 52, the gap must be open at least 1.5 mm to allow material to exit but not more than 2.5 mm which would produce material size too large for use as NHSM fuel in cement kilns

The extruder 20 heats, compresses, mixes, grinds, and crushes the infectious waste as the waste moves there through. The resulting final product is a decontaminated material that is unrecognizable and is suitable for use as fuel in a cement kiln. The operating conditions of the extrusion process are selected so that the final product is substantially homogeneous fluff, as opposed to a briquette form, wherein the final product has been compressed to about an 8 to 1 ratio in relation to the infectious waste that is inserted in the feed hopper 16. The amount of the thermomechanical heat is controlled in particular by the rotational speed of the extruder auger members 40 and 42 and the orientation of the reversible auger flight sections 62 and 72.

In operation of the decontamination system 10, after the infectious waste has passed through the feed mixer 18, the waste will then pass into the first compression chamber 44 of the extruder 20, which also begins operation when the feed mixer 18 is activated. The variable speed motor 22 will then be set to rotate the extruder augers 40 and 42 at a pre-set rpm level of 90 RPM+−5%. Greater speeds would melt the plastic and less would not create enough heat for disinfection.

The thermomechanical disinfection process begins in the first compression chamber 44. The counter rotating augers 40 and 42 grind and begin to homogenize the waste. At the same time, the waste is forced downstream into the more aggressive auger sections 58. Here the waste is forced against the front side 60 of the first friction plate 50 until it is small enough to pass through the gap under the first friction plate 50. Once past the first friction plate, the waste encounters the auger sections 62 which have reverse direction flights, and force the waste back against the opposite, back side 68 of the first friction plate 50, thus increasing the thermomechanical heat being developed.

The waste stream is further homogenized and volume reduced inside of the second compression chamber 46 where the same process in the first combustion chamber 44 is repeated and the waste receives additional thermomechanical heat and volume reduction. This process will ensure that all material passing through the extruder 20 will be decontaminated. The internal thermal sensors connected to the process control unit 12 measure the internal temperatures of the extruder 20.

The pre-set temperature is preferably over 205° F., more preferably 250° F., and most preferably over 300° F. Reversing one or both of the normally reverse pitch auger sections 62 and 72 controls the process temperature. Reversing these flight sections is a simple matter of swapping the right and left hand flight sections. Which side of the auger shaft these flights are mounted determines the direction of the auger flights.

Once the material has been treated by the system 10, the material is sold and supplied to one or more cement kiln owners or operators where the material is used as fuel to maintain the high temperature fire in the cement kiln. As already noted, the consumption of the waste material as fuel in the cement kiln results in an ash or residue that becomes part of the generated cement mixture. Thus, unlike in a landfill, the waste is completely disposed of and the waste provider actually derives income from the cement kiln owners.

Although the invention has been disclosed in terms of a preferred embodiment and variations thereon, it will be understood that numerous other variations and modifications could be made thereto without departing from the scope of the invention as set forth in the following claims.

1. A thermal friction extruder for use in an infectious waste treatment system comprising:
(i) a housing defining a material flow passage, said housing having an upstream end and a downstream end;
(ii) first and second interleaved counter-rotatable augers disposed in said housing and passing through said flow passage from said upstream end to said downstream end; each of said augers having a root and a plurality of flight sections on said root, wherein said first auger has flights that are opposite in direction to the flights of said second auger;
(iii) at least a first compression chamber for receiving infectious waste material at said upstream end portion of said housing;
(iv) at least a first friction plate in said housing and defining a first end of said first compression chamber, said friction plate being positioned over said roots of said first and second augers between first forward pitch flight sections and first reverse pitch flight sections, said friction plate being configured to form a gap between said friction plate and said auger roots which causes waste material on a first side of said friction plate to be urged by said first forward pitch flights into said friction plate, thereby imparting frictional heat to said waste material, and wherein said reverse pitch flight sections are configured such that waste material will be urged in an upstream direction toward a second side of said friction plate but will not be prevented from traveling overall toward said downstream end of said housing;
(v) an outlet for discharging treated waste from said extruder at said downstream end of said housing; and
(viii) a variable speed motor coupled to said first and second augers for rotating said first and second augers in opposite directions with respect to one another;
wherein, said friction plate gap and the speed of said motor are selected so that the resulting treated medical waste has an increased BTU value and is of small aggregate consistency such that said material can be classified as NHSM and used as fuel in a cement kiln.

2. The extruder of claim 1, wherein the flights of said reverse pitch sections are angled at between 15 and 35 degrees about a vertical axis toward said upstream end of said housing.

3. The extruder of claim 1, wherein a second friction plate is disposed in said housing, said second friction plate defining a second end of said first compression chamber and being positioned over said roots of said first and second augers between second forward pitch flight sections and second reverse pitch flight sections, said second friction plate being configured to form a gap between said second friction plate and said auger roots which causes waste material on a first side of said second friction plate to be urged by said second forward pitch flights into said second friction plate, thereby imparting frictional heat to said waste material, and wherein said second reverse pitch flight sections are configured such that waste material will be urged in an upstream direction toward said second side of said second friction plate but will not be prevented from traveling overall toward said downstream end of said housing, wherein said gap of said second friction plate is selected to open at least 1.5 mm to allow material to exit but not more than 2.5 mm which would produce material size too large for use as NHSM in cement kilns.

4. The extruder of claim 3, wherein said reverse pitch auger sections are removable and can be selectively replaced to become additional forward pitch auger sections to reduce the amount of heat imparted to the waste material in said chamber during operation.

5. The extruder of claim 1, wherein a series of said compression chambers, friction plates and reverse pitch auger sections is provided.

6. The extruder of claim 5, wherein each of the reverse pitch flight sections is independently replaceable with a forward pitch flight section to facilitate more precise control of the heat imparted to the waste material in the compression chambers during operation.

7. The extruder of claim 1, wherein the reverse pitch auger sections of both auger members are removable and can be changed to forward pitch auger sections by swapping the augers on which said reverse pitch auger sections are mounted.

8. The extruder of claim 1, further including a process control unit for controlling operation of said extruder motor in response thereto.
